SEQUENCE LISTING (2 sequences)

SEQ ID NO: 1 - protein, 595 aa, Artificial Sequence
  Description of Artificial Sequence/note = synthetic construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 2 - DNA, 6450 bp, Artificial Sequence
  Description of Artificial Sequence/note = synthetic construct
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

(19) United States
(12) Patent Application Publication
(10) Pub. No.: US 2002/0164581 A1
(43) Pub. Date: Nov. 7, 2002

(54) Methods for identifying compounds that do not attenuate the protective effects of estrogen

(76) Inventor: Chawnshang Chang, Pittsford, NY (US)

Correspondence Address: Gwendolyn D. Spratt, Esq., NEEDLE & ROSENBERG, P.C., The Candler Building, Suite 1200, 127 Peachtree Street, N.E., Atlanta, GA, 30303-1811, US

(21) Appl. No.: 10/096,710

(22) Filed: Mar. 12, 2002

Related U.S. Application Data
(60) Provisional application No. 60/275,229, filed on Mar. 12, 2001.

(51) Int. Cl. C12Q 1/00; G01N 33/53; G01N 33/567

(52) U.S. Cl. 435/4; 435/7.2

(57) ABSTRACT

Disclosed are compositions and methods for determining whether a compound attenuates the protection an estrogen confers on a population of cells.

This application claims priority to U.S. patent application Ser. No. 60/275,229 filed on Mar. 12, 2001 for “Methods for Identifying Compounds that do not Attenuate the Protective Effects of Estrogen”, which application is herein incorporated by reference in its entirety.

### I. BACKGROUND OF THE INVENTION

Pre-menopausal women have a significantly lower incidence of cardiovascular disease than do men and post-menopausal women (Bush, Ann. Acad. Sci. 592:263-271, 1990). The differences in the occurrence of this disease between genders and among women of different menopausal states may be attributed to the action of endogenous estrogen. This view is supported by the demonstration that estrogen replacement therapy decreases the risk of cardiovascular disease in post-menopausal women (Bush, Ann. Acad. Sci. 592:263-271, 1990; Stampfer et al., N. Engl. J. Med. 325:756-762, 1991; Green et al. Balliere's Clin. Endocrinol. Metab. 7:95-112, 1993).

The cardiovascular benefits of estrogen result from estrogen's ability to favorably impact plasma lipids, preserve endothelium-mediated vasodilatation, and produce anti-platelet and antioxidative effects (Stampfer, et al., N. Engl. J. Med. 325:756-762, 1991, Nathan et al., Annu. Rev. Pharmacol. Toxicol. 37:477-515, 1997).

Disclosed are methods and compositions that allow one to identify beneficial activities of estrogen and related molecules.

### II. SUMMARY OF THE INVENTION

In accordance with the purposes of this invention, as embodied and broadly described herein, this invention, in one aspect, relates to compositions and methods for determining whether a compound attenuates the protection an estrogen confers on a population of cells.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

The accompanying drawings, which are incorporated in and constitute a part of this specification, illustrate several embodiments of the invention and together with the description, serve to explain the principles of the invention.

FIGS. 1A-1C are bar graphs depicting the surviving fraction of bAECs exposed to various concentrations of E₂ (0 to 10 μM) for 24 hours prior to oxidative challenge. The bar represented by “0 nM” represents a control experiment in which cells were incubated with 0.05% DMSO for 24 hours. The remaining bars represent experiments in which bAECs treated with E₂ were rinsed with PBS twice and challenged with 1 mM paraquat for 3 hours at 37° C. The inserts show the results obtained when E₂-pretreated cells were subjected to 1 mM homocysteine plus 25 μM Cu²⁺ for 3 hours or 10 μg/ml of lysophosphatidylcholine (LPC) for 60 minutes at 37 C. In FIG. 1B, the chemical antioxidant effect of E₂ was investigated by adding 1 nM-10 μM E₂ to cells during the 3 hour paraquat exposure. FIG. 1C represents studies of the chemical and cell-mediated effects of estrogen. bAECs were pretreated with E₂, followed by E₂ supplementation during subsequent paraquat exposure as described above. After oxidative challenge, the relative cell survival was measured by an MTT assay, and is expressed as “surviving fraction” relative to cells that were not oxidatively challenged. Data shown are. * and ** indicate P<0.05 and P<0.01 respectively, vs. estrogen-untreated cells (0 nM).

FIG. 2 is a line graph that illustrates that pretreatment with E₂ induces a right-shift in the paraquat dose-response curve (the concentration of paraquat is graphed along the x-axis in mM, and the fraction of surviving bEACs is graphed along the y-axis. Filled circles represent cells that received no E₂ treatment and open circles represent cells that were treated with 1 nM E₂ for 24 hours. Cells were pretreated with 1 nM of E2 for 24 hours and then challenged with increasing doses of paraquat for 3 hours. Control cells were incubated with 0.05% DMSO and subjected to the same oxidative challenge. The relative cell survival after paraquat challenge was measured by the MTT assay and expressed as surviving fraction. Mean±SEM from 6-8 wells of cells are given. ** indicates P<0.01 vs. estrogen-untreated cells.

FIG. 3 is a bar graph illustrating the differential effect of E₂-related steroids and SERMs on endothelial cell protection. Cells were incubated with 1 nM each of E₂, nafoxidine, diethylstilbestrol (DES), estrone, 4-hydroxytamoxifen (4-OH-Tam), and raloxifene (Ral) at 37° C. for 24 hours, rinsed with PBS, and challenged with 1 mM of paraquat for 3 hours. The surviving fraction of cells, after challenge, was determined as described herein. Mean±SEM from 4-8 wells of cells is shown. ** indicates P<0.01 vs. the steroid-untreated cells.

FIG. 4 is a line graph illustrating that 4-OH-Tam selectively blocks E₂-mediated endothelial protection. The potential effect of SERMs on endothelial function was further investigated by pretreatment of bAECs for 24 hours with various concentrations of 4-OH-Tam (open circles) or Ral (closed circles). To investigate a potential interaction between SERMs and E2, some cells were given both 1 nM of E2 and various concentrations of 4-OH-Tam (open triangles) or Ral (closed triangles) during the 24 hour pretreatment. After pretreatment, SERM-induced changes in oxidative resistance were examined by subjecting the cells to 1 mM of paraquat for 3 hours. The surviving fraction of cells was determined after oxidated challenge and is shown as the mean±SEM from 4-8 wells of cells. * and ** indicate P<0.05 and P<0.01 vs. 1 nM E₂-pretreated cells (0 nM of SERMs), respectively.

FIGS. 5A-5D are bar graphs illustrating that E₂ pretreatment induces the activities of AOE. Cells were plated in 10-cm tissue culture plates and then exposed to different concentrations of E₂ for 24 hours. The activities of SOD (FIG. 5A), CAT (FIG. 5B), GPX (FIG. 5C), and GR (FIG. 5D) were examined in the resultant cell lysate following the conditions described below. Data shown are mean±SEM from 4-6 plates of cells. Note that a brief E₂ exposure (30 minutes) had no immediate effect to induce AOE, indicating that the observed AOE induction after E2 pretreatment may be due to a chronic effect of this sex hormone.

FIG. 6 is a bar graph that illustrates that 4-OH-Tam selectively blocks E₂-mediated AOE induction. Cells were incubated with 1 nM E₂, with or without each SERM for 24 hours. Subsequently, the cells were harvested and the activities of SOD, CAT, GPX, and GR were assayed as described below. Mean±SEM from 4-6 plates of cells are given. ** indicates the difference between E₂-treated and 4-OH Tam/E₂-cotreated cells at P<0.01. Note that pretreatment of cells with 1 μM of 4-OH-Tam or Ral alone caused no detectable change in AOE, and that the inhibition of AOE induction by 4-OH-Tam correlated with the suppression of E₂-mediated oxidative protection by this SERM.

FIGS. 7A-7C are line graphs illustrating the protection E₂ confers on female, but not male, bAECs. In FIG. 7A, the relative number of female (closed circles) and male (open circles) bAECs is shown following exposure to various concentrations of E₂ (1 nM-10 μM) for 24 hours and challenge with paraquat (1 mM) for three hours. The relative cell survival was measured by an MTT assay and is expressed as surviving fraction compared to cells without paraquat insult. The data shown in FIG. 7B were obtained when bAECs were incubated without E₂ for 24 hours, then with E₂ at the indicated concentrations before challenge with 1 mM paraquat (still in the presence of E₂) for 3 hours. The surviving fraction was measured using the MTT assay. Cells from five female and four male animals were tested. Cells from each animal were plated in 4 wells. Means±SEM were derived from the averages of the tested wells. “a” indicates P<0.05 vs. male cells at the tested E₂ concentration, t-test; “b” indicates P<0.05 vs. 0 nM control, ANOVA and Tukey test; “c” indicates P<0.05 vs. 0 nM control for both female and male cells, ANOVA and Tukey test. In FIG. 7C, E₂-mediated oxidative protection was confirmed by cell count. The experimental design was the same as for the experiments reported in FIG. 7A. However, after the 48-hour recovery period, cells in each well were harvested (by exposure to trypsin), resuspended in a small volume of medium, and counted with a hematocytometer. The averages derived from cells of two female and two male donors are shown. Cell counts among groups pretreated with various doses of E₂ without subsequent paraquat exposure were comparable, averaging 3.1×10⁵ per well.

FIGS. 8A and 8B are line graphs illustrating that thermal preconditioning provides protection against paraquat injury in both male and female bAECs. Male and female bAECs were heated to 43 C for 30 minutes (filled circles) and then allowed to recover for 24 hours. Control cells were cultured at 37 C (open circles). The cells were then challenged with 0.2-2.0 mM paraquat, and the surviving fraction was measured after a 48-hour recovery period. Cells from four female and three male animals were included. Cells from each animal were plated in 4 wells. Means±SEM. a: P<0.05 vs. 37 C control, t-test.

FIGS. 9A-9D are bar graphs illustrating the differential effect of E₂ on the induction of antioxidant enzyme activities in male and female bAECs. The activities of superoxide dismutase (FIG. 9A), catalase (FIG. 9B), glutathione peroxidase (FIG. 9C) and glutathione reductase (FIG. 9D) were measured. Both male and female bAECs were incubated with 1 nM E₂ at 37° C. for 24 hours and then harvested for the enzyme assays. Cells from three females and three males were included. Cells from each animal were assayed twice. The averages were used to calculate means±SEM. a: P<0.05 vs. 0 nM control, t-test.

FIG. 9 is a pair of panels showing the distribution of DCFH fluorescence in female and male bAECs upon paraquat exposure. Cells were pretreated with 1 nM E₂ for 24 hours and then harvested and incubated with dichlorofluorescein diacetate. The cells were then exposed to 1 mM paraquat for 15 minutes (dotted line) or 45 minutes (solid line), and the fluorescence of oxidized DCFH was detected by flow cytometry. Compared with untreated controls (dashed line), paraquat exposure shifted DCFH fluorescence to the right.

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that for any particular value given herein, that values is also disclosed as “about” the value. For example, if “5%” is disclosed, it is understood that “about 5%” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

B. Methods of Using the Compositions

Estrogen has been shown to have beneficial effects on cardiovascular disease. Estrogen has also been linked to the increased antioxidant activity. For example, at supraphysiological levels (5-50 μM), 17β-estradiol (E₂) protects against low density lipoprotein modification or oxidative stress-induced cell injury in vitro, potentially via its chemical, antioxidant activity (Maziere et al., Atherosclerosis 89:175-182, 1991; Behl et al., Biochem. Biophys. Res. Commun. 216:473-482, 1995). However, is it unclear that E₂ confers similar chemical protection at physiological concentrations. Other studies indicate that in vivo administration of E₂ up-regulates the antioxidant enzyme (AOE) activities such as superoxide dismutase (SOD), catalase (CAT), glutathione peroxidase (GPX), or glutathione reductase (GR) in aortae, ovaries, and erythrocytes (Ghanam et al., Biochem. Biophys. Res. Comm. 249:858-864, 1998; Singh and Pandey, J. Exp. Biol. 36:421-423, 1998; Massafra et al., J. Clin. Endocrinol. Metab. 82:173-175, 1997), but it is not known whether this enzymatic induction is the result of a direct or indirect E₂ effect.

Disclosed herein bovine aortic endothelial cells (bAECs) were used to examine the direct effect of physiological concentrations of 17β-estradiol (E2) on oxidative protection and antioxidant enzymes and to screen for compounds that inhibit the protective effect that estrogens, such as E2, confer on cells. Accordingly, the invention features methods of screening for compounds that inhibit the protective effect (e.g. the cardioprotective effect) of estrogens, such as E2, estrone, and estriol on various cell types. The method also can identify compounds that do not inhibit a protective effect of an estrogen, as well as those that potentiate one or more of their effects. Moreover, by testing a range of concentrations, the methods can be used to determine the concentration at which (and above which) the compound inhibits one or more of the protective effects conferred by an estrogen. Any compound can be screened (e.g., one can screen small molecules, peptides, or proteins, regardless of whether these molecules are new or have already been identified as useful therapeutic agents). Among the compounds that should be screened are those that act as either agonists or antagonists of an estrogen receptor. For example, tamoxifen and active metabolites thereof, such as 4-hydroxytamoxifen (4-OH-Tam), can be screened.

The findings disclosed herein, including the observations that E2 induces cell-mediated oxidative protection at physiological levels and that protection can be attenuated by certain compounds, are significant because low density lipoprotein (LDL) peroxidation and the excessive uptake of oxidized LDL by macrophages play important roles in atherogenic processes. E2, estrone, and estriol at 5-50 μM inhibit LDL oxidation by copper ions, monocytes, or endothelial cells, whereas testosterone has no effect (Maziere et al., Atherosclerosis 89:175-182, 1991). In addition, E2 inhibits oxidized LDL- or hydrogen peroxide-induced cell death when administered in the μM range (Behl et al., Biochem. Biophys. Res. Commun. 216:473-482, 1995; Negre-Salvayer et al., Atherosclerosis 99:207-217, 1993). However, the effective concentrations of E2 used in these prior studies are several orders of magnitude higher than plasma E2 levels (0.4-4.0 nM) in women even during their reproductive years. Thus, these studies cannot answer the question or whether or not E2 acts as a direct antioxidant and thereby confers oxidative protection when applied at physiological levels.

The methods can be carried out in vitro or in vivo by examining cells, such as endothelial cells, ovarian cells, and erythrocytes, which are positively impacted by (i.e. protected from an insult by) an estrogen such as E2. For example, the methods can be carried out using cultured endothelial cells (e.g. bovine aortic endothelial cells or endothelial cells from another mammalian species (e.g. porcine, canine, equine, or human endothelial cells obtained from an artery or vein)). In addition, the methods can be carried out using cell types that do not normally express receptors for estrogen molecules but that have been genetically modified to do so (e.g. the methods can be carried out with cells that contain one or more exogenous nucleic acid sequences that encode an estrogen receptor). Cells that express estrogen receptors are available to those of ordinary skill in the art from, for example, the American Type Culture Collection (Manassas, Va.). Alternatively, they can be isolated from tissues (e.g. bovine, porcine, equine, or human tissues) according to standard procedures (see, e.g. the Examples below for isolation of bAECs).

The cells selected can be examined in a number of ways. For example, one can assay the cells for their ability to survive or to proliferate, as well as for their ability to express proteins whose expression or activity is upregulated when the cells are exposed to, and positively impacted by (i.e. protected from an insult by), an estrogen. Assays useful in evaluating cell survival or proliferation are well known in the art. Many of the proteins that are upregulated when cells are exposed to, and positively impacted by, an estrogen are also known and can be assayed by methods routinely used in the art. For example, the activity of antioxidant enzymes (AOEs) is upregulated in bAECs that have been exposed to E2. AOEs are well known to those of ordinary skill in the art and include, enzymes which catalyze the destruction of free radicals including peroxidases such as glutathione peroxidase (GSHPX) which acts on H2O2 and such as organic peroxides, including catalase (CAT) which acts on H₂O₂, superoxide dismutase (SOD) which disproportionates O2H2O2; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; and GPX.

Accordingly, in one embodiment, the invention features a method for determining whether a compound attenuates the protection an estrogen confers on a population of cells. The method can be carried out by exposing the cells to the compound and the estrogen, treating the cells with an agent that would, in the absence of the estrogen, damage the cells (e.g., an agent that generates a reactive oxygen species), and evaluating the survival of the cells. If there is a decrease in the number of cells that survive, relative to that within a population of cells that has not been exposed to the compound, then the compound is one that attenuates the protection the estrogen confers on that population of cells. This information is useful to patients and physicians because it will allow them to identify and administer compounds that will not attenuate the protective effects of estrogen.

In other embodiments, the method is carried out by evaluating the expression of, or the activity of, AOEs in the cells. In this case, if there is a decrease in the expression of, or the activity of, AOEs, relative to that within a population of cells that has not been exposed to the compound, then the compound is one that attenuates the protection the estrogen confers on that population of cells.

In other embodiments, the method is carried out by evaluating the expression of, or the activity of, nitric oxide synthase in the cells. E2 at physiological concentrations induces nitric oxide synthase in endothelial cells from different origins, including bovine aorta, human aorta, human umbilical vein, and lamb pulmonary artery. Thus, if there is a decrease in the expression of, or the activity of, nitric oxide synthase, relative to that within a population of cells that has not been exposed to the compound, then the compound is one that attenuates the protection the estrogen confers on that population of cells.

A compound “attenuates” the protective effect of an estrogen when it inhibits any activity, such as antioxidant related activity or cell protective activity regulated by an estrogen by as little as 5% (e.g. 5, 10, 25, 50, 70, 80, 90% or more). For example, if a compound inhibits the activity of an AOE by 5% or more, that compound attenuates the protection an estrogen confers on a population of cells. When the cells are assessed in terms of their survival or the rate at which they proliferate, a compound that attenuates the protective effect of an estrogen is one that reduces the survival of the cells within the tested population, or the rate at which they proliferate, by at least 5% (e.g. 5, 10, 25, 50, 70, 80, 90% or more).

The disclosed methods include methods for making libraries from induced cells and looking for differential gene expression. For example, the disclosed methods and compositions have shown that in the presence of estrogen receptor and estrogen, genes involved in protection from oxidative cell stress are activated. The disclosed methods and compositions can therefore, be used to, for example, screen cells having an estrogen receptor in the presence of oxidative stress and estrogen to analyze which genes are activated and which genes are suppressed. Likewise, these disclosed methods and compositions can be used for analyzing the differential expression effect of molecules, such as SERMs both in and out of the presence of estrogen or estrogen like compounds.

When present at supra-physiological concentrations (i.e., ˜μM), estrogen displays a free radical-scavenging effect. Disclosed herein, female bovine aortic endothelial cells that were pretreated with physiological concentrations (1-10 nM) of 17β-estradiol for 24 hours were more resistant to paraquat, homocysteine, and lysophosphatidylcholine. This indicated that estrogen, such as 17β-estradiol, produced a protective effect on the cells. However, when the cells were treated with 1 nM to 1 μM of 4-hydroxytamoxifen and raloxifene, the benefit conferred by 17β-estradiol was reduced. More specifically, the addition of 4-hydroxytamoxifen (10 nM) during the estradiol pretreatment period suppressed oxidative protection, and the addition of 1 μM of raloxifene during the pretreatment period slightly inhibited the protective effect. It is relevant to the conferred oxidative protection that estradiol, at 1-10 nM, induced the activities of several enzymes: superoxide dismutase, catalase, glutathione reductase, and glutathione peroxidase. The ability of estradiol to induce these enzymes was blocked by 4-hydroxytamoxifen, but not raloxifene.

Taken together, these studies establish that estradiol, at physiological concentrations, protects endothelial cells by inducing protective mechanisms in the cells, such as those that increase in the amount of, or the activity of, antioxidant enzymes. The finding of a selective suppression of estrogen's benefits by 4-hydroxytamoxifen is of particular interest in the context of cardiovascular disease risk because it suggests a need to evaluate the long-term use of compounds, such as selective estrogen receptor modulators (SERMs), in pre-menopausal women or post-menopausal women receiving estrogen replacement therapy. If the compound is one that inhibits or attenuates a protective effect mediated by an estrogen (e.g., the expression of, or the activity of, an antioxidant enzyme), it is undesirable for that reason. To the contrary, if the compound is one that does not inhibit or attenuate a protective effect mediated by an estrogen (here again, these effects can be assessed by examining the expression of, or the activity of, an antioxidant enzyme), that compound is a more desirable candidate for administration.

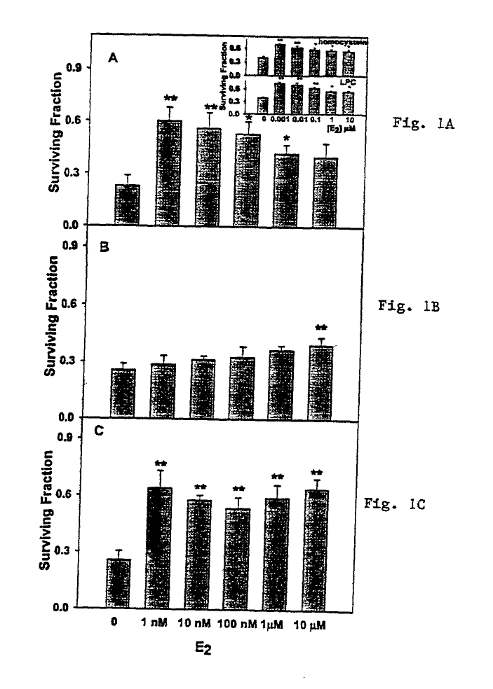

Studies have suggested that estrogen may have “antioxidant” activities in vivo. Compared to controls, LDL isolated from post-menopausal women receiving estrogen replacement therapy is more resistant to chemical oxidation (Sack et al., Lancet 343:269-270, 1994). This direct protection of LDL against oxidative modification depends on the presence of plasma acyltransferase activity and may involve the incorporation of E2 into LDL through esterification (Shwaery et al., Circulation 95:1378-1385, 1997). Disclosed herein, (see Examples 1-8), notable endothelial protection was observed when E2 was applied to the cells at a highly elevated concentration (10 μM) and in the absence of serum during an oxidative insult (see FIG. 1B). The oxidative protection acquired by cells after pretreatment with E2 for 24 hours may be due to better incorporation of E2 into lipid bilayers or lipid-protein complexes, which would increase cellular resistance to the toxicity of reactive oxygen species generated during subsequent oxidative challenges. However, the lack of a definitive dose-dependent response in E2 pretreatment-mediated oxidative protection (FIG. 1A) indicates that such a chemical, antioxidant protection may be minimal in cells incubated with E2 for a prolonged duration of 24 hours. It is more likely that certain intracellular defense mechanism(s) are induced by E2 during the prolonged pretreatment, and that the induction of these mechanisms may be feedback suppressed by the increasing concentrations of this hormone.

E2 facilitates the preservation of vascular endothelial functions (Keaney et al., Circulation 89:22510-2259, 1994). Blood vessels from ovariectomized female miniature swine fed a high-fat diet exhibit impaired relaxation to endothelium-dependent vasodilators (e.g., bradykinin or substance P), but not to the endothelium-independent dilators (e.g., nitroglycerin). This suggests a dysfunction of nitric oxide production/release. Recent studies have demonstrated that E2 at physiological concentrations induces nitric oxide synthase in endothelial cells from different origins, including bovine aorta, human aorta, human umbilical vein, and lamb pulmonary artery (Hayashi et al., Biochem. Biophys. Res. Commun, 214:847-855, 1995; Hishikawa et al., FEBS Lett. 360:291-293, 1995; Weiner et al., Proc. Natl. Acad. Sci. USA 91:5212-5216, 1994; Lantin-Hermoso et al., Am. J. Physiol. 273:L119-L126, 1997; MacRitchie et al., Circ. Res. 81:355-362, 1997; Kleinert et al., Hypertension 31:582-588, 1998). Since E2 induces endothelial nitric oxide synthase activities, its role in the maintenance and/or preservation of endothelial functions is strengthened. The optimal concentration of E2 for endothelial nitric oxide synthase induction is thought to be within the range of 1-10 nM, and the magnitude of induction may decrease upon the increasing concentrations of E2 (Hayashi et al., Biochem. Biohphys. Res. Commun. 214:847-855, 1995; MacRitchie et al., Circ. Res. 81:355-362, 1997). These observations agree with the findings disclosed herein that E2-induced endothelial protection results from the induction of certain protective enzymes.

The present finding that SOD was induced by E2 is consistent with the observation that ICI164384, a pure estrogen receptor antagonist, reverses the beneficial effect of E2, suggesting that E2 may act through a receptor-dependent pathway to detoxify superoxide anions, and an induction of SOD may be involved. Differences in the employed cell types and/or treatment protocols (aortic explants were used by Cathapermal et al. (J. Cardiovasc. Pharmacol. 31:499-505, 1998)) may contribute to the small discrepancy in the effective concentration of E2 to induce SOD and cytoprotection between the previous and present studies.

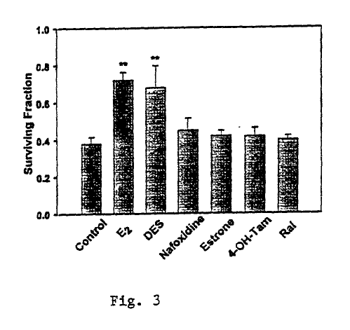

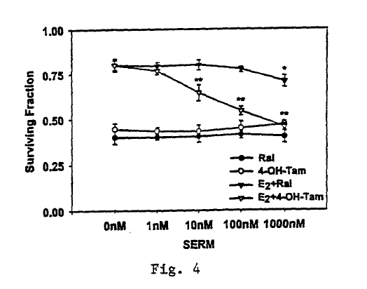

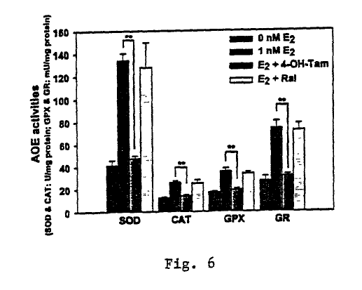

It is well recognized that SERMs may act as either partial estrogen receptor antagonists or agonists on different tissues (Bryant and Dere, Proc. Soc. Exp. Boil. Med. 217:45-52, 1998). Neither 4-OH-Tam, an active metabolite of tamoxifen, nor Ral at 1 nM-1 μM provided a significant cytoprotection in female bAEC (FIGS. 3 and 4), indicating that these SERMs do not have E2-like activity to confer oxidative resistance. The addition of 4-OH-Tam during E2 pretreatment selectively blocked the induction of oxidative protection and AOE by E2 (FIGS. 4 and 6), whereas Ral, which is structurally and functionally different from 4-OH-Tam, exhibited only a mild or negligible inhibitory effect. This indicates that 4-OH-Tam, but not Ral, may act as an effective E2 antagonist in bAEC. While SERMs are proposed as chemopreventive agents in women with a high breast cancer risk, prescribing SERMs to pre-menopausal women for this purpose creates a scenario in which pharmacological levels of SERMs coexist with physiological concentrations of E2. Disclosed herein, when 4-OH-Tam in tamoxifen users accumulates to levels interfering with E2's normal function, endothelial protection could be compromised. The disclosed methods can identify SERMs or any molecule that interferes with the protective effects of estrogen, thus allowing clinicians to weigh the protective effects and the negative effects of a particular molecule being administered, for example, in a preventative chemotherapy role.

Using cardiac mortality or cardiac disease-resulted hospital admission as the criterion, several clinical trials showed that tamoxifen does not exert a detrimental effect on the heart (McDonald and Stewart, BMJ 303:435-437, 1991; Rutqvist and Mattsson, J. Natl. Cancer Inst. 85:1398-1406, 1993; McDonald et al., BMJ 311:977-980, 1995; Fisher et al., J. Natl. Cancer Inst. 90:1371-1388, 1998; Constantino et al., J. Natl. Cancer Inst. 89:776-782, 1997), but only a low percentage and in some cases, none of the participants, were premenopausal women. Therefore, it remains uncertain whether tamoxifen may have non-life threatening side effects in, especially pre-menopausal, women.

Recently, Saphner et al. (J. Clin. Oncol. 9:286-294, 1991) reported a greater rate of vascular events, such as venous and arterial thrombi, in pre-menopausal women receiving tamoxifen and chemotherapy than in pre-menopausal women receiving chemotherapy alone. Emerging evidence indicates still other undesirable effects of tamoxifen. Tamoxifen at high doses causes asymptomatic prolongation of the QT interval on electrocardiogram in humans (Trump et al., J. Natl. Cancer Inst. 84:1811-1816, 1992; Pollack et al., Clin. Cancer Res. 3:1109-1115, 1997). This electrocardiogram abnormality resolves after discontinuation of tamoxifen treatment, suggesting that tamoxifen interferes with electric conductance in the myocardium. Interestingly, E2 has been shown to prevent high K+-induced intracellular Ca++ loading or hypercontracture in cardiomyocytes, and tamoxifen abolishes this protective effect (Jovanovic et al., Ann. Thorac. Surg. 66:1658-1661, 1998).

Tamoxifen also interferes with E2's ability to induce nitric oxide synthase in endothelial cells (Hayashi et al., Biochem. Biophys. Res. Commun. 214:847-855, 1995). As shown herein, an active metabolite of tamoxifen, 4-OH-Tam, suppressed E2-mediated oxidative protection in endothelial cells (see FIG. 4). It is noteworthy that the minimal dose (10 nM) of 4-OH-Tam to suppress E2 protection is achievable in breast cancer patients receiving tamoxifen therapy (Daniel et al., J. Endocrinol. 83:401-408, 1979). These observations make clear that there is a need to assess the health-related effects of tamoxifen and/or other SERMs in pre-menopausal women or post-menopausal women receiving estrogen replacement therapy.

In summary, the results disclosed here show that the optimal protection by E2 in bAECs is within physiological levels of this sex hormone. As E2 concentration increases, oxidative protection conferred by E2 pretreatment decreases, suggesting that the beneficial effect of E2 is not be due to its chemical, antioxidant activity. Instead, the induction of cellular defense mechanisms, such as those mediated by AOEs, are likely to play a more active role. The observation that 4-OH-Tam, but not Ral, blocks the beneficial effect of E2 demonstrates a feasibility of using bAECs, as well as cells expressing estrogen receptor, to study the differential effects of SERMs in the presence of physiological concentrations of E2.

The disclosed compositions can be used in a variety of ways as research tools. For example, the disclosed compositions, such as the cells and reagents can be used to study the effect of a given molecule on the beneficial effects of estrogen.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular estrogen molecule, such as 17β-estradiol, is disclosed and discussed and a number of modifications that can be made to a number of molecules including the estrogen molecule, such as 17β-estradiol, are discussed, specifically contemplated is each and every combination and permutation of estrogen molecule, such as 17β-estradiol, and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Homology/Identity

It is understood that one way to define any known variants and derivatives or those that might arise, of the disclosed genes and proteins herein is through defining the variants and derivatives in terms of homology to specific known sequences. For example disclosed are cells that have been engineered to express an estrogen receptor. It is understood that any molecule having homology to an estrogen receptor that produces a functional estrogen receptor could be used. For example, specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level. The sequences of estrogen receptors and known allelic variants and functional variants are available at for example, the sequence database Genbank, and are herein specifically incorporated by reference for their sequence information known when this application was filed.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based, including for example the nucleic acids that encode, for example, the estrogen receptor, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Sequences

There are a variety of sequences related to the estrogen receptor gene having the following non-limiting but exemplary set of the Genbank Accession Numbers: AF258449, AF258450, X03635, NM_{—}000125, XM_{—}045967, M69297, M69296, M12674, AF061181, AF258451, Z75126, U68068, and U68067 including alternatively spliced sequences, and these sequences and others are herein incorporated by reference in their entireties as well as for individual subsequences contained therein.

One particular estrogen receptor protein and mRNA sequence are set forth in SEQ ID NOs:1 and 2 and having Genbank accession number X03635 are used herein, as an example, to exemplify the disclosed compositions and methods. It is understood that the description related to these sequences is applicable to any sequence related to the estrogen receptor unless specifically indicated otherwise. Those of skill in the art understand how to resolve sequence discrepancies and differences and to adjust the compositions and methods relating to a particular sequence to other related sequences. Primers and/or probes can be designed for any estrogen sequence given the information disclosed herein and known in the art. It is understood that variants of the estrogen receptor that function as an estrogen receptor are disclosed herein to be used in the disclosed methods. Protein variants in general and related to the estrogen receptor are discussed herein.

4. Nucleic Acid Delivery

In the methods described above which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

5. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

6. Peptides

a) Protein Variants

As discussed herein there are numerous variants of the estrogen receptor that can be used in the disclosed compositions and methods. In addition, to the known functional estrogen receptor allelic variants there are derivatives of the estrogen receptor which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following sets and are referred to as conservative substitutions: Ala and ser; Arg, lys, and gin; Asn, gln and his; Asp and glu; Cys and ser; Gln, asn, and lys; Glu and asp; Gly and pro; His, asn; and gln; Ile, leu; and val; Leu, ile; and val; Lys, arg; and gln; Met, Leu; and ile; Phe, met; leu; and tyr; Ser and thr; Thr and ser; Trp and tyr; Tyr, trp; and phe; and Val, ile; and leu.

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

7. Compositions Identified by Screening with Disclosed Compositions and Methods

Disclosed are compositions and methods for identifying compositions, which do not inhibit or attenuate the beneficial effects of estrogen, such as an increase in antioxidant activity. It is understood that the disclosed compositions and methods can also identify ranges of dosages and predictive levels to be used in vivo. For example, a given molecule, for example, a SERM may attenuate the beneficial effects of estrogen at a particular concentration, but not attenuate the beneficial effects of estrogen or reduce the attenuation at another concentration. This latter concentration, identified using the disclosed methods and compositions represents a target concentration for therapeutic purposes. Thus, if for example, the molecule could be administered in vivo at a concentration which still produces the beneficial pharmacological effect without reducing the beneficial estrogen activities, this would be preferred. It is understood that disclosed herein, are the molecules and ranges so identified using the disclosed compositions and methods.

8. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include cells, estrogen, or an estrogen related molecule, and reagents which could be used to test a given molecule or molecules for inhibition of the beneficial activities of estrogen as disclosed herein. For example, disclosed is a kit for assessing a molecules effect on the beneficial activity of estrogen.

D. Methods of Making the Compositions

The compositions disclosed herein and the compositions necessary to perform the disclosed methods can be made using any method known to those of skill in the art for that particular reagent or compound unless otherwise specifically noted.

Disclosed are processes for making the compositions and kits as well as making the intermediates leading to the compositions and kits. There are a variety of methods that can be used for making these compositions and kits, such as synthetic chemical methods and standard molecular biology methods and combining the various reagents contained within the kits. It is understood that the methods of making these and the other are specifically disclosed.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Primary Culture of bAECs

Primary cultures of bAECs were established from five female beef cattle (ranging in age from 1 to 2 years old) according to the method of Fenselau and Mello (Cancer Res. 36:3269-3273, 1976). Pooled cells were routinely maintained under humidified air (5% CO₂) at 37° C. in Dulbecco's modified medium supplemented with 10% fetal bovine serum (FBS). Cells from the second to the fifth passages were used in these studies. Within these passage numbers, >95% cells were stained positively by factor VIII antibody (A0082, DAKO, Carpinteria, Calif., USA) following the method of Diglio et al. (Lab. Invest. 46:554-563, 1982).

In the studies in which male and female bAECs were compared, batches of bAECs were independently established from 5 female and 4 male beef cattle (ranging in age from 1 to 2 years old) according to the method of Fenselau and Mello (Cancer Res. 36:3269-3273, 1976). Otherwise, the cells were treated and assessed as described in the paragraph above.

### 2. Example 2

E₂ Pretreatment and Oxidant Challenge

Prior to E₂ pretreatment, cells were grown in phenol red-free DMEM supplemented with 10% dextran/charcoal-stripped FBS for at least 1 passage (3-4 days). Phenol red-free Dulbecco's modified medium was used to avoid potential estrogenic interference. Serum steroid deprivation was conducted according to the conditions described by Yeh et al. (Proc. Natl. Acad. Sci. USA 95:5527-5532, 1998). The E₂ level in the stripped serum was assayed using an [¹²⁵I] 17β-estradiol radioimmunoassay kit (TKE21, Diagnostic Products Corporation, Los Angeles, Calif.). The E₂ levels in the different batches of deprived sera were consistently undetectable (<0.1 nM). The cells grown in E₂-free environments were plated onto Falcon 24-well Primariae™ plates at a density of 5×10⁴ cells/well. E₂ in dimethylsulfoxide (DMSO) was added to each well at final concentrations ranging from 1 nM to 10 μM 3 hours after plating, and the cells were then incubated for 24 hours (a duration commonly used for the studies of the genomic effects of E₂) at 37° C. Control cells were treated with a corresponding amount of DMSO (a final concentration 0.05%). These dosages of E₂ did not adversely affect cell morphology or growth. However, when reaching a concentration ≧100 μM, E₂ significantly inhibited endothelial cell growth. Therefore, the E₂ effect at these highly elevated concentrations was not examined.

Prior to oxidative challenge, E₂-containing media was removed, and the cells were rinsed with PBS twice and re-fed with serum-free fresh medium. Subsequently, paraquat was added to the cells at the indicated doses (0.1-2.0 mM) for 3 hours. The oxidative challenge was terminated by aspiration of the oxidant-containing medium and quickly rinsing the cells twice with PBS. The cells were allowed to recover for 48 hours in medium supplemented with 10% regular FBS to exclude the possibility that different concentrations of E₂ interfere with the cellular recovery processes. The 48 hour recovery time allows dying cells to detach from the substratum, resulting in a highly reduced background.

Cell survival after oxidative challenge was assessed with a 3-(4,5-dimethylthiazol-2-yl) 2,5 diphenyltetrazolium bromide (MTT) assay in which the mitochondrial dehydrogenase activity of viable cells reduces MTT to a blue formazan product (Carmichael et al. Cancer Res. 47:936-942, 1987). Our prior conditions were employed (Si et al., Endocrine in press, 2001). Briefly, cells were treated with 1/10 volume of MTT solution (5 mg/ml in PBS) and incubated at 37° C. for 2 hours. The supernatant was then removed, and 600 μl of lysis buffer (50% dimethyl formamide, 5% sodium dodecyl sulphate, 0.35 M acetic acid, 50 mM HCl) was added to each well. Following cell lysis, the absorbency was measured at 570 nm and used as an indicator of relative cell survival. The surviving fraction was calculated as compared to the cells without paraquat challenge. Data derived from the MTT assay were found to be comparable with those obtained from cell counts (Si et al., Endocrine in press, 2001).

Where male and female bAECs were examined, the LD₅₀ of paraquat in cells from both genders was about 0.1-1.0 mM. Two experimental groups were created. In the first group, E₂ was added only during the 24-hour pretreatment period. In the second group, E₂ was added only during the 3-hour paraquat exposure. After paraquat exposure, both groups of cells were washed twice with PBS and allowed to recover for 48 hours in fresh medium plus 10% regular FBS to exclude the possibility that different concentrations of E₂ interfere with the cellular recovery processes.

To determine whether intrinsic inducible defense mechanisms exist in both male and female bAECs, thermal preconditioning was applied to induce the beneficial heat shock response. Briefly, cells originating from both geners were heated at 43° C. for 30 minutes and allowed to recover at 37° C. for 24 hours to induce this beneficial adaptation, which has been shown to protect cells against oxidative damage. The control cells were maintained at 37° C. for this period of time. The cells were then challenged with different doses of paraquat (0.2-2.0 mM) and left to recover for 48 hours before being subjected to the MTT assay.

### 3. Example 3

Preparation of Cell Lysates for Enzyme Assays

Cells pretreated with different concentrations of E₂ for 24 hours were harvested in a solution containing 50 mM Tris-HCl (pH 7.4), 1 mM EDTA, 2 μg/ml pepstatin A, 10 μg/ml benzamidine, and 10 μg/ml trypsin inhibitor. The cells were then lysed on ice by sonication at a setting of 50 mW, with 3×15 set cycles using a Cole-Parmer Ultrasonic Homogenizer (Chicago, Ill.). Protein concentrations in the samples were determined by the Bradford method with bovine serum albumin as a standard (Bradford, Anal. Biochem. 72:248-254, 1976).

### 4. Example 4

Antioxidant Enzyme Assays

SOD activity was determined by a method based on the ability of SOD to suppress epinephrine auto-oxidation (Misra and Fridovich, J.Biol. Chem. 247:3170-3175, 1972). One unit of SOD activity is defined as the amount of protein that inhibits the oxidation of epinephrine by 50%. Briefly, 100 μl of 10×-diluted cell homogenate (about 10 μg protein) was added to 900 μl of carbonate buffer (0.1 M NaHCO₃, 0.1 M Na₂CO₃, 0.1 mM EDTA, pH 10.2) containing 0.7 mM of epinephrine. The absorbency of the mixture was measured for 4 minutes at 480 nm using a Hitachi U-2000 spectrophotometer (Hitachi, San Jose, Calif.).

CAT activity was determined by the method of Aebi (Methods Enzymol. 105:121,-126, 1984). One unit of CAT activity is defined as the amount of protein that degrades 1 μmole of H₂O₂ per minute. Initially, 10 μl of absolute ethanol was mixed with 100 μl of cell homogenates and pre-incubated at 4° C. for 30 minutes. Following the addition of 10 μl Triton X-100 (reduced form), a 100 μl aliquot (about 100 μg of protein) of the resultant mixture was next added to 500 μl of hydrogen peroxide (66 mM) and 400 μl of phosphate buffer (0.1 M NaH₂PO₄, 0.1 M Na₂HPO₄, 0.1 mM EDTA, pH 7.0). The absorbency of the mixture was monitored for one minute at 240 nm spectrophotometrically. The molar extinction coefficient of 43.6 M/cm was used to determine the catalase activity.

GPX activity was determined by a modified method of Flohe and Gunzler (Methods Enzymol. 105:114-121, 1984). One unit of GPX activity is defined as the amount of protein that oxidizes 1 nmole of β-NADPH per minute. In this assay, 100 μl of cell homogenate (about 100 μg protein) was added to 800 μl of phosphate buffer (0.1 M NaH₂PO₄, 0.1 M Na₂HPO₄, 0.1 mM EDTA, pH 7.0) containing 12.5 mM glutathione (reduced form, GSH), 0.2 mM β-nicotinamide adenine dinucleotide phosphate (reduced form, β-NADPH), and 0.34 U glutathione reductase. This mixture was pre-incubated at 37 C for 10 minutes and then 100 μl of 12 mM t-butyl hydroperoxide was added. The absorbency of the mixture was determined at 340 nm for 3 minutes. The molar extinction coefficient of 6.22 mM/cm was used to determine the activity of GPX.

GR activity was determined by a modified method of Carlberg and Mannervik (Methods Enzymol. 11 3:484-490, 1985). One unit of GR activity is defined as the amount of protein that oxidizes 1 nmole of β-NADPH per minute. Briefly, 50 μl of 2 mM β-NADPH in 10 mM Tris buffer (pH 7.0) was added to a cuvette containing 50 μl of 20 mM glutathione (oxidized ofrm, GSSG) in phosphate buffer (0.1 M NaH₂PO₄, 0.1 M Na₂HPO₄, 0.1 mM EDTA, pH 7.0) and 800 μl of phosphate buffer. Following the addition of cell homogenate (about 100 μg protein/100 μl) the final mixture was measured at 340 nm for 3 minutes. The molar extinction coefficient of 6.22 mM/cm was used to determine the activity of GPX.

For flow cytometric analysis (after E₂ treatment), female and male bAECs were trypsinized, rinsed, and resuspended in PBS containing 2% BSA at a density of 5×10⁵ cells/ml. The cells were loaded with dichlorofluorescin diacetate (Molecular Probes, Eugene, Oreg.) (dissolved in DMSO) at 5 μg/ml for 15 minutes followed by paraquat insult (1 mM) at room temperature in the dark. At the designated times, aliquots were withdrawn, placed on ice, and the fluorescence of oxidized DCFH was analyzed by a FACSCalibur™ flow cytometer (Becton-Dickinson, San Jose, Calif.) equipped with an argon laser. A collection filter for the green emission of oxidized DCFH (530/30 nm) was used. Fluorescence signal was collected in log mode and analyzed using Cellquest™ software (Becton-Dickinson).

Relative cell surviving fraction and enzyme activities were reported as mean±SEM (n≧4). Differences between control and treated groups were analyzed by unpaired Student's t-test or ANOVA and post hoc Scheffe's procedures.

### 5. Example 5

Incubation of Cells with E₂ Augments Oxidative Resistance

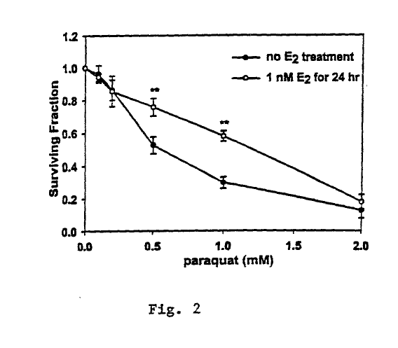

Paraquat is a herbicide that generates intracellular superoxide radicals upon metabolic conversion. Exposure of bAECs to this oxidant for 3 hours at 37° C. caused a dose-dependent cell killing. Compared with the untreated control, cells pretreated with E₂ for 24 hours displayed an enhanced resistance to the toxic effect of paraquat (FIG. 1A). E₂-pretreated cells also gained survival benefit upon subsequent challenge with lysophosphatidylcholine (10 μg/ml, 60 min) or homocysteine/Cu²⁺ (1 mM/25 μM, 3 hours) (FIG. 1A, inserts), both of which, like paraquat, trigger the formation of reactive oxygen species. The LD₅₀ of paraquat toxicity in cells pretreated with 1 nM of E₂ was elevated from about 0.5 mM (in the untreated control) to about 1 mM (FIG. 2). This observation of a right shift in the dose-response curve of paraquat toxicity upon E₂ pretreatment further confirms the beneficial effect of estrogen. It is noteworthy that endothelial protection evoked by E₂ was not shown in a dose-dependent manner. The E₂-evoked protection was optimal in cells pretreated with E2 at 1-10 nM, and gradually declined in magnitude with increasing dosages of E₂ at about 1 μM (FIG. 1A). Thus, physiological concentrations of E₂ may selectively augment cellular resistance to reactive oxygen species.

### 6. Example 6

A Short Incubation with E₂ Exerted Minimal Effects on Oxidative Protection

To test the possibility that oxidative protection is due to the chemical, antioxidant effect of residual E₂ in the pretreated cells, E₂ was directly added to the cells during paraquat exposure, and then monitored the survival after the challenge. If the protection observed (and described above) is the result of the chemical, free radical-scavenging effect of E₂, it would seem reasonable that including E₂ during the oxidative challenge would confer greater endothelial protection than that afforded by E₂ pretreatment. However, contrary to this speculation, only the highest tested dosage of E₂ (10 μM) during oxidative challenge defended cells against paraquat-mediated killing (FIG. 1B). In addition, the combination of E₂ pretreatment and E₂ inclusion during subsequent paraquat exposure altered the pattern of cytoprotection (FIG. 1C). The biphasic protection acquired after the combined E₂ administration mimics a superimposition of the protection afforded by E₂ pretreatment (FIG. 1A) and that afforded by E₂ inclusion during oxidative exposure (FIG. 1B). These findings indicate that E₂provides bAECs with two types of oxidative protection: a cell-mediated protective mechanism that is maximally evoked by E₂ at physiological levels (FIG. 1A) and a chemical, antioxidant effect that is efficiently expressed only at a highly elevated concentration of E₂, such as 10 μM (FIG. 1B).

However, this does not exclude the possibility that E₂ inclusion during oxidative challenge activates cellular antioxidant defenses via a rapid “non-genomic” pathway to provide endothelial protection. To test this possibility, bAECs were incubated with 1 nM to 10 μM E₂ for 30 minutes, rinsed with PBS, and then subjected to paraquat challenge. Such a brief preincubation of cells with estrogen was not effective in providing oxidative protection. This confirms that survival benefits obtained when 10 μM E₂ is included during paraquat exposure (FIG. 1B) are more likely attributable to the chemical, free radical-scavenging effect of available E₂ in the medium.

### 7. Example 7

Selective Estrogen Receptor Modulators (SERMs) Influence E₂-Mediated Oxidative Protection

To determine whether E₂-related steroids and/or SERMs, such as 4-hydroxytamoxifen (4-OH-Tam) and raloxifene (Ral) also enhance cellular oxidative resistance, bAECs were incubated with 1 nM of diethylstilbestrol, estrone, nafoxidine, 4-OH-Tam, or Ral for 24 hours, and then rinsed with PBS and challenged with paraquat. FIG. 3 shows that only cells pretreated with E₂ (or its agonist, diethylstilbestrol) acquired significant protection against subsequent oxidative challenge. The other tested compounds neither positively nor negatively influenced oxidative resistance. As shown in FIG. 4, pretreatment of bAECs with Ral or 4-OH-Tam at more elevated concentrations 10 nM-1 μM) still failed to confer detectable protection against paraquat-mediated cytotoxicity.. This indicates that these SERMs do not exhibit an estrogen-agonist activity to activate cellular antioxidant defenses. Interestingly, 10 nM-1 μM of 4-OH-Tam when added simultaneously with E₂ (1 nM) during the pretreatment significantly blocked estrogen-induced oxidative protection (FIG. 4). Ral at 1-100 nM, in contrast, did not affect E₂-induced oxidative protection. At the highest tested dose (1 μM), Ral displayed only a slight inhibitory effect on E₂ protection.

### 8. Example 8

SERMs Influence Et₂-Mediated AOE Induction

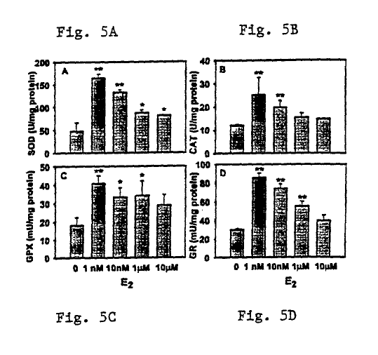

bAECs incubated with 1 nM-10 μM E₂ for 24 hours displayed significant increases in the activities of all four of the enzymes tested (FIGS. 5A-5D) indicating that physiological concentrations of E₂ indicating E₂'s effect on antioxidant defense mechanism(s). Optimal induction was achieved by 1-10 nM of E₂. The activities of SOD, CAT, GR, and GPX in cells pretreated with E₂ (1 nM) were elevated by approximately 4-fold, 2-fold, 3-fold, and 2-fold, respectively, relative to their activities in untreated cells. The enzymatic induction was less with increasing concentrations of E₂ and correlated with the decline in oxidative protection acquired by cells pretreated with E₂ at the same concentrations (see FIG. 1A). AOE induction by E₂ was abolished when cyclohexamide was including during the E₂ pretreatment period, which indicates that enzyme induction involves new protein synthesis. In addition, a brief incubation (30 minutes) of bAECs with 1 nM E₂ did not affect the activities of AOEs, indicating that a more prolonged pretreatment with E₂ is needed to evoke AOE expression. Moreover, the inclusion of 1 μM of 4-OH-Tam, but not Ral, during E₂ (1 nM) pretreatment significantly decreased estrogen's capability to evoke AOE activities (FIG. 6). This correlates with the observed negative influence of 4-OH-Tam on E2-mediated oxidative protection (FIG. 4). Taken together, these findings show that E₂, at physiological concentrations, confers oxidative protection via AOE induction.

### 9. Example 9

The Effect of Estrogen on Female and Male bAECs Exposed to Oxidative Injury

To study the difference between male and female cells in their response to E₂, bAECs from male and female animals were treated with various concentrations of E₂ for 24 hours and then challenged with paraquat (1,1′-dimethyl-4,4′-bipyridinium dichloride, methyl viologen). Paraquat was used because it generates the superoxide anion O2 in living cells. Preliminary experiments showed that E₂ (1 nM to 10 μM) does not significantly affect growth in either male or female bAECs. Thus, E₂ has no effect on normal endothelial cell proliferation when applied for 24 hours at those concentrations (E₂ at concentrations ≧100 μM for 24 hours did suppress the growth of both male and female bAECs). Accordingly, E₂ ranging in concentration from 1 nM to 10 μM was used in these studies.

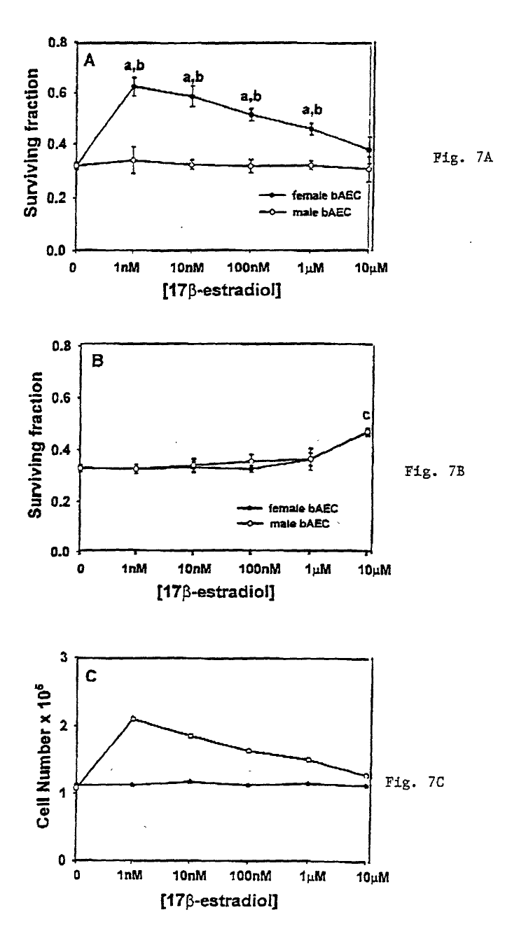

A marked augmentation of oxidative resistance, as assessed by an MTT survival assay, was observed in female bAECs pretreated with 1-10 nM E₂ for 24 hours and then challenged with paraquat. The magnitude of this protection gradually decreased as the concentrations of E₂ were elevated to 0.1-10 μM (P<0.05 vs. 1 nM, ANOVA and Tukey test; see FIG. 7A, filled circles). In contrast, no protection was seen against paraquat challenge in male bAECs pretreated with E₂ at any of the tested concentrations (P=not significant, ANOVA; see FIG. 7A, open circles). These results were confirmed by cell count after the challenge (FIG. 7C). It appears that E₂ at physiological concentrations induces or activates in the female, but not in the male, bAEC intrinsic protective mechanisms capable of defending against the intracellularly formed superoxide anion.

### 10. Example 10

Direct Antioxidant Activity of E₂

One postulate is that the direct free radical scavenging activity of E₂ is concentration-dependent and readily available to both male and female bAECs when E₂ is present during the oxidant challenge. To differentiate between this direct form of chemical protection and cell-mediated antioxidative activities, E₂ was added directly to both male and female cells during a 3-hour paraquat exposure. As predicted, the presence of E₂ at elevated concentrations (≧1-10 μM) in the culture media provided mild but notable oxidant protection in both male and female bAECs against paraquat-induced killing (FIG. 7B). While free radical scavenging by E₂ could account for this protection at μM levels (FIG. 7B), the added E₂ could also hastily trigger some “low sensitivity” cellular defense systems in both male and female bAECs. To eliminate the possibility that E₂ acts through a fast, non-genomic signal pathway to enhance cellular defense, E₂ was added to bAECs at corresponding concentrations for 30 minutes only. Following the removal of E₂ by extensive wash, these short-term E₂-treated cells were similarly exposed to paraquat and their viability was assayed. No protection by E₂ as observed in either male or female endothelial cells receiving such a brief estrogenic exposure. This indicates that E₂ can exert an effective free radical scavenging activity against intracellular superoxide only at levels far exceeding the physiological concentrations. Thus, the observed difference between male and female bAECs in response to E₂ is due to the induction of cellular antioxidant defense systems.

### 11. Example 11

Heat Preconditioning Protects Both Male and Female Cells Against Oxidative Injury

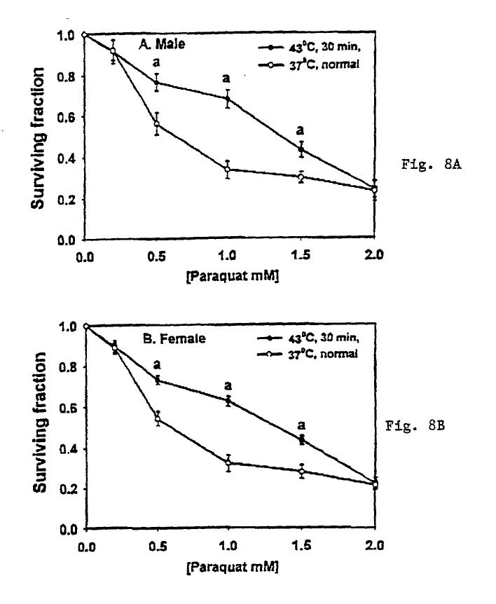

Induction of a heat shock response may provide protection to cardiovascular cells against oxidative insults, such as exposure to hydrogen peroxide or ischemic or reperfusion injuries. To test whether male and female bAECs are equally responsive to a non-selective inducer, such as heat preconditioning, cells originating from both genders were preheated to 43° C. for 30 minutes, allowed to recover for 24 hours at 37° C., and then challenged with different doses of paraquat (0.2-2.0 mM). Compared with the unheated control, the preheated male and female cells were significantly more resistant to paraquat (FIGS. 8A and 8B). This indicates that both sexes contain similar defense systems inducible by heat shock and that cultured male bAECs can adapt to environmental stresses.

### 12. Example 12

Differential Induction of AOEs in Male and Female bAECs

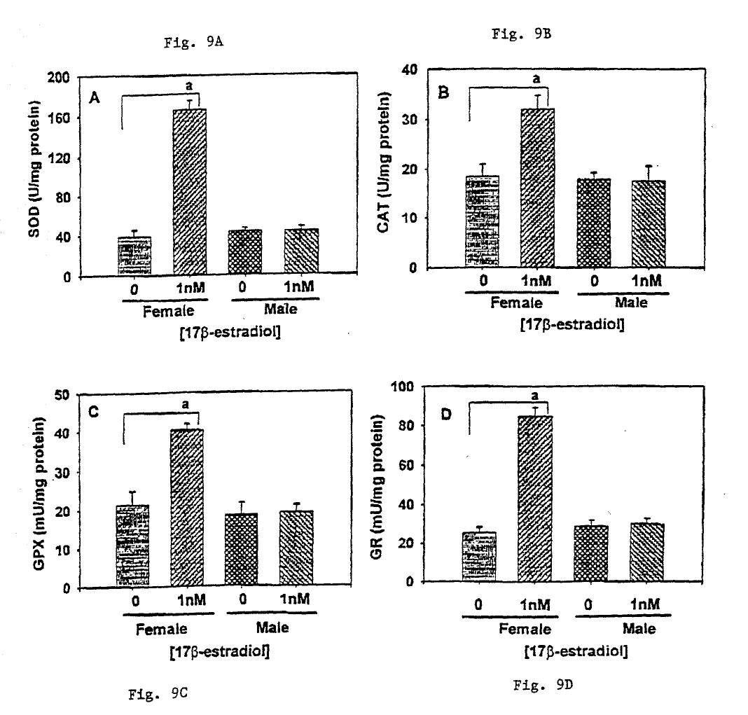

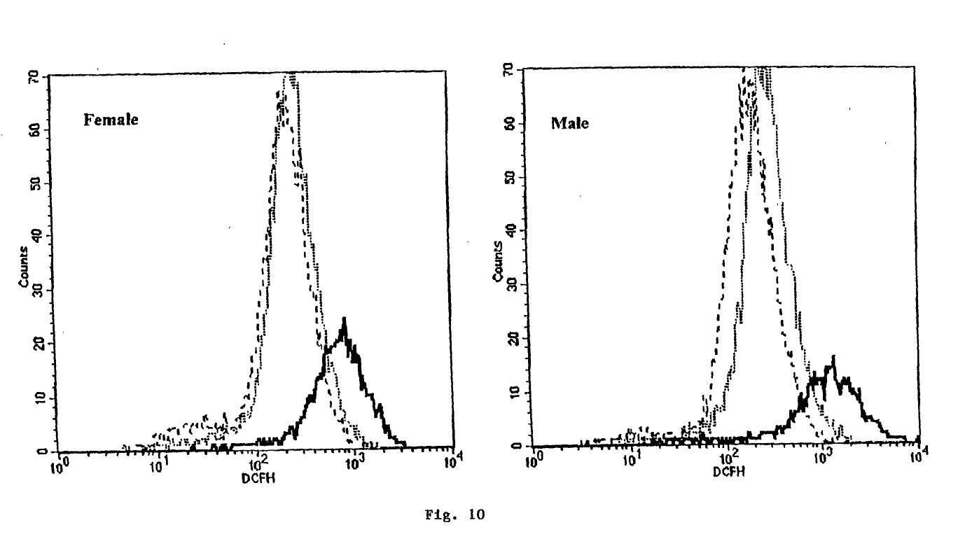

These experiments were conducted to explore the protective mechanisms that are differentially activated by physiological concentrations of E₂ in male and female bAECs. The activities of AOEs, including SOD, CAT, GPX, and GR are readily induced by E₂. The optimal E₂ concentration of AOE induction is about 1-10 nM, and the magnitude of AOE induction in female bAECs gradually decreased upon increasing concentrations of E₂. This reverse dose response cure coincides with the observed female cell survival following paraquat challenge (FIG. 7A), indicating the AOEs play important roles in the E₂-induced oxidative protection of female bAECs. Following a 24 hour, 1 nM, E₂ treatment, female bAECs showed a significant elevation of all of the four AOE activities tested. The activities of SOD, CAT, GPX, and GR were increased by about 4-fold, 2-fold, 3-fold, and 2-fold, respectively, in female bAECs (FIGS. 9A and 9B). In contrast, the activities of these enzymes were not induced in male bAECs incubated with 1 nM of E₂. The disclosed methods include methods for making libraries from induced cells and looking for differential gene expression.

### 13. Example 13

Reduced Levels of Reactive Oxygen Species (ROS) in E2-Pretreated Cells

SOD can dismutate the superoxide radical to hydrogen peroxide, which is subsequently detoxified by CAT or GPX. In theory, and induction of SOD, CAT, and GPX by E₂ as observed in female bAECs may enhance the catabolism of ROS. This theory was tested by comparing the level of ROS in female and male bAECs during subsequent oxidant exposure. Following E₂ treatment for 24 hours, cells were loaded with dichlorofluorescein diacetate, which penetrates their plasma membranes and is converted to 2′,7′-dichlorofluorescein (DCFH) by intracellular esterases. This nonfluorescent compound, once oxidized by ROS such as hydrogen peroxide, turns to a highly fluorescent derivative. FIG. 10 shows that the basal level of DCFH oxidation in E₂-pretreated female and male bAECs was similar. Upon exposure to 1 mM of paraquat, DCFH oxidation increased. The fluorescence emitted by oxidized DCFH was significantly augmented in cells after a 45-minute exposure to paraquat. Interestingly, DCFH oxidation was lower in female (the mean fluorescence intensity was 795) than in male cells (the mean fluorescence intensity was 1323). This gender difference in DCFH oxidation was confirmed using bAECs from other donors (the mean DCFH fluorescence after a 45-minute paraquat exposure was 1002 and 1581 for a separate set of female and male cells, respectively). In the absence of E₂ pretreatment, DCFH oxidation by paraquat, however, was comparable between female and male bAECs.

F. Sequences

SEQ ID NO:1 Genbank accession number X03635. for Human protein sequence of an estrogen receptor

MTMTLHTKASGMALLHQIQGNELEPLNRPQLKIPLERPLGEVYLDSSKPAVYNYPEGAAYEFNAAA
AANAQVYGQTGLPYGPGSEAAAFGSNGLGGFPPLNSVSPSPLMLLHPPPQLSPFLQPHGQQVPYYLE
NEPSGYTVREAGPPAFYRPNSDNRRQGGRERLASTNDKGSMAMESAKETRYCAVCNDYASGYHY
GVWSCEGCKAFFKRSIQGHNDYMCPATNQCTIDKNRRKSCQACRLRKCYEVGMMKGGIRKDRRG
GRMLKHKRQRDDGEGRGEVGSAGDMRAANLWPSPLMIKRSKKNSLALSLTADQMVSALLDAEPPI
LYSEYDPTRPFSEASMMGLLTNLADRELVHMINWAKRVPGFVDLTLHDQVHLLECAWLEILMJGLV
WRSMEHPVKLLFAPNLLLDRNQGKCVEGMVETFDMLLATSSRFRMMNLQGEEFVCLKSJILLNSGV
YTFLSSTLKSLEEKDHIHRVLDKITDTLIHLMAKAGLTLQQQHQRLAQLLLILSHIRHMSNKGMEHL
YSMKCKNVVPLYDLLLBMLDAHRLHAPTSRGGASVBETDQSHLATAGSTSSHSLQKYYITGEAEGF
PATV

SEQ ID NO:2 Genbank accession number X03635. for Human mRNA sequence of an estrogen receptor

1 gagttgtgcc tggagtgatg ttaagccaa tgtcagggca aggcaacagt ccctggccgt

61 cctccagcac ctttgtaatg catatgagct cgggagacca gtacttaaag ttggaggccc

121 gggagcccag gagctggcgg agggcgttcg tcctgggagc tgcacttgct ccgtcgggtc

181 gccggcttca ccggaccgca ggctcccggg gcagggccgg ggccagagct cgcgtgtcgg

241 cgggacatgc gctgcgtcgc ctctaacctc gggctgtgct ctttttccag gtggcccgcc

301 ggtttctgag ccttctgccc tgcggggaca cggtctgcac cctgcccgcg gocacggacc

361 atgaccatga ccctccacac caaagcatct gggatggccc tactgcatca gatccaaggg

421 aacgagctgg agcccctgaa ccgtccgcag ctcaagatcc ccctggagcg gcccctgggc

481 gaggtgtacc tggacagcag caagcccgcc gtgtacaact accccgaggg cgccgcctac

541 gagttcaacg ccgcggccgc cgccaacgcg caggtctacg gtcagaccgg cctcccetac

601 ggccccgggt ctgaggctgc ggcgttcggc tccaacggcc tggggggttt cecccactc

661 aacagcgtgt ctccgagccc gctgatgcta ctgcacccgc cgccgcagct gtcgcctttc

721 ctgcagcccc acggccagca ggtgccctac tacctggaga acgagcccag cggctacacg

781 gtgcgcgagg ccggcccgcc ggcattctac aggccaaatt cagataatcg acgccagggt

841 ggcagagaaa gattggccag taccaatgac aagggaagta tggctatgga atctgccaag

901 gagactcgct actgtgcagt gtgcaatgac tatgcttcag gctaccatta tggagtctgg

961 tcctgtgagg gctgcaaggc cttcttcaag agaagtattc aaggacataa cgactatatg

1021 tgtccagcca ccaaccagtg caccattgat aaaaacagga ggaagagctg ccaggcctgc

1081 cggctccgca aatgctacga agtgggaatg atgaaaggtg ggatacgaaa agaccgaaga

1141 ggagggagaa tgttgaaaca caagcgccag agagatgatg gggagggcag gggtgaagtg

1201 gggtctgctg gagacatgag agctgccaac ctttggccaa gcccgctcat gatcaaacgc

1261 tctaagaaga acagcctggc cttgtccctg acggccgacc agatggtcag tgccttgttg

1321 gatgctgagc cccccatact ctattccgag tatgatccta ccagaccctt cagtgaagct

1381 tcgatgatgg gcttactgac caacctggca gacagggagc tggttcacat gatcaactgg

1441 gcgaagaggg tgccaggctttgtggatttg accctccatg atcaggtcca ccttctagaa

1501 tgtgcctggc tagagatcct gatgattggt ctcgtctggc gctccatgga gcacccagtg

1561 aagctactgt ttgctcctaa cttgctcftg gacaggaacc agggaaaatg tgtagagggc

1621 atggtggaga tcttcgacat gctgctggct acatcatctc ggttccgcat gatgaatctg

1681 cagggagagg agtttgtgtg cctcaaatct attattttgc ttaattctgg agtgtacaca

1741 tttctgtcca gcaccctgaa gtctctggaa gagaaggacc atatccaccg agtcctggac

1801 aagatcacag acactttgat ccacctgatg gccaaggcag gcctgaccct gcagcagcag

1861 caccagcggc tggcccagct cctcctcatc ctctcccaca tcaggeacat gagtaacaaa

1921 ggcatggagc atctgtacag catgaagtgc aagaacgtgg tgcccctcta tgacctgctg

1981 ctggagatgc tggacgccca ccgcctacat gcgcccacta gccgtggagg ggcatccgtg

2041 gaggagacgg accaaagcca cttggccact gcgggctcta cttcatcgca ftccttgcaa

2101 aagtattaca tcacggggga ggcagagggt ttccctgcca cagtctgaga gctccctggc

2161 tcccacacgg ttcagataat ccctgctgca ttttaccctc ateatgcacc actttagcca

2221 aattctgtct cctgcataca ctccggcatg catccaacac caatggcttt ctagatgagt

2281 ggccattcat ttgcttgctc agttcttagt ggcacatctt ctgtcttctg ttgggaacag

2341 ccaaagggat tccaaggcta aatctttgta acagctctct ttcccccttg ctatgttact

2401 aagcgtgagg attcccgtag ctcttcacag ctgaactcag tctatgggtt ggggctcaga

2461 taactctgtg catttaagct acttgtagag acccaggcct ggagagtaga cattttgcct

2521 ctgataagca ctttttaaat ggctctaaga ataagccaca gcaaagaatt taaagtggct

2581 cctttaattg gtgacttgga gaaagctagg tcaagggttt attatagcac cctcttgtat

2641 tcctatggca atgcatcctt ttatgaaagt ggtacacctt aaagctttta tatgactgta

2701 gcagagtatc tggtgattgt caattcactt ccccctatag gaatacaagg ggccacacag

2761 ggaaggcaga tccctagtt ggccaagact tattttaact tgatacactg cagattcaga

2821 gtgtcctgaa gctctgcctc tggctttccg gtcatgggtt ccagttaatt catgcctccc

2881 atggacctat ggagagcaac aagttgatct tagttaagtc tccctatatg agggataagt

2941 tcctgatttt tgtttttatt tttgtgttac aaaagaaagc cctccctccc tgaacttgca

3001 gtaaggtcag cttcaggacc tgttccagtg ggcactgtac ttggatcttc ccggcgtgtg

3061 tgtgccttac acaggggtga actgttcact gtggtgatgc atgatgaggg taaatggtag

3121 ttgaaaggag caggggccct ggtgttgcat ttagccctgg ggcatggagc tgaacagtac

3181 ttgtgcagga ttgttgtggc tactagagaa caagagggaa agtagggcag aaactggata

3241 cagttctgag cacagccaga cftgctcagg tggccctgca caggctgcag ctacctagga

3301 acattccttg cagaccccgc attgcctttg ggggtgccct gggatccctg gggtagtcca

3361 gctcttattc atttcccagc gtggccctgg ttggaagaag cagctgtcaa gttgtagaca

3421 gctgtgttcc tacaattggc ccagcaccct ggggcacggg agaagggtgg ggaccgttgc

3481 tgtcactact caggctgact ggggcctggt cagattacgt atgcccttgg tggtttagag

3541 ataatccaaa atcagggttt ggtttgggga agaaaatcct cccccttcct cccccgcccc

3601 gttccctacc gcctccactc ctgccagctc atttccttca atttcctttg acctataggc

3661 taaaaaagaa aggctcattc cagccacagg gcagccttcc ctgggccttt gctctctag

3721 cacaattatg ggttacttcc tttttcttaa caaaaaagaa tgtftgattt cctctgggtg

3781 accttattgt ctgtaattga aaccctattg agaggtgatg tctgtgttag ccaatgaccc

3841 aggtagctgc tcgggcttct cttggtatgt cttgtttgga aaagtggatt tcattcattt

3901 ctgattgtcc agttaagtga tcaccaaagg actgagaatc tgggagggca aaaaaaaaaa

3961 aaaaagtttt tatgtgcact taaatttggg gacaatttta tgtatctgtg ttaaggatat

4021 gcttaagaac ataattcttt tgttgctgtt tgtttaagaa gcaccttagt ttgtttaaga

4081 agcaccttat atagtataat atatafttit ttgaaattac attgcttgtt tatcagacaa

4141 ttgaatgtag taattctgtt ctggatttaa tttgactggg ttaacatgca aaaaccaagg

4201 aaaaatattt agtttttttt tttttttttg tatacttttc aagctacctt gtcatgtata

4261 cagtcattta tgcctaaagc ctggtgatta ttcatttaaa tgaagatcac atttcatatc

4321 aacttttgta tccacagtag acaaaatagc actaatccag atgectattg ttggatattg

4381 aatgacagac aatcttatgt agcaaagatt atgcctgaaa aggaaaatta ttcagggeag

4441 ctaattttgc ttttaccaaa atatcagtag taatattttt ggacagtagc taatgggtca

4501 gtgggttctt tttaatgttt atacttagat tttcttttaa aaaaattaaa ataaaacaaa

4561 aaaaatttct aggactagac gatgtaatac cagctaaagc caaacaatta tacagtggaa

4621 ggttttacat tattcatcca atgtgtttct attcatgtta agatactact acatttgaag

4681 tgggcagaga acatcagatg attgaaatgt tcgcccaggg gtctccagea actttggaaa

4741 tctctttgta tttttacttg aagtgccact aatggacagc agatattttc tggctgatgt

4801 tggtattggg tgtaggaaca tgatttaaaa aaaaaactet tgcctctgct tteecccact

4861 ctgaggcaag ttaaaatgta aaagatgtga tttatctggg gggcteaggt atggtgggga

4921 agtggattca ggaatctggg gaatggcaaa tatattaaga agagtattga aagtatttgg

4981 aggaaaatgg ttaattctgg gtgtgcacca aggttcagta gagtccactt ctgceetgga

5041 gaccacaaat caactagctc catttacagc catftctaaa atggcagctt cagttctaga

5101 gaagaaagaa caacatcagc agtaaagtcc atggaatagc tagtggtctg tgtttcttt

5161 cgceattgec tagcttgccg taatgattft ataatgccat catgcagcaa ttatgagagg

5221 ctaggtcatc caaagagaag accctatcaa tgtaggttgc aaaatctaac ccctaaggaa

5281 gtgeagtctt tgatttgatt tecctagtaa ccttgcagat atgtttaacc aagccatagc

5341 ccatgccttt tgagggctga acaaataagg gacttactga taatttactt ttgatcacat

5401 taaggtgttc tcaccttgaa atettataca ctgaaatggc cattgattta ggccactggc

5461 ttagagtact ccttcccetg catgacactg attacaaata ctttcctatt catactttcc

5521 aattatgaga tggactgtgg gtactgggag tgatcactaa caccatagta atgtetaata

5581 ttcacaggca gatctgcttg gggaagctag ttatgtgaaa ggcaaataaa gtcatacagt

5641 agctcaaaag geaaccataa ttctctttgg tgcaagtctt gggagegtga tetagattac

5701 actgcaccat tcecaagtta atcccctgaa aacttactct caactggagc aaatgaactt

5761 tggtcccaaa tatccatctt ttcagtagcg ttaattatgc tctgtttcca actgcatttc

5821 ctttccaatt gaattaaagt gtggcctcgt ttttagtcat ttaaaattgt fttctaagta

5881 attgctgcct ctattatggc acttcaattt tgcactgtct tttgagattc aagaaaaatt

5941 tctattcatt tttttgcatc caattgtgcc tgaactttta aaatatgtaa atgctgccat

6001 gttccaaacc catcgtcagt gtgtgtgttt agagctgtgc accctagaaa caacatactt

6061 gtcccatgag caggtgcctg agacacagac ccctttgcat tcacagagag gtcaftggtt

6121 atagagactt gaattaataa gtgacattat gccagtttct gttctctcac aggtgataaa

6181 caatgctftt tgtgcactac atactcttca gtgtagagct cttgttffat gggaaaaggc

6241 tcaaatgcca aattgtgttt gatggattaa tatgccctt tgccgatgca tactattact

6301 gatgtgactc ggttttgtcg cagctttgct ttgtttaatg aaacacactt gtaaacctct

6361 tttgcacttt gaaaaagaat ccagcgggat gctcgagcac ctgtaaacaa ttttctcaac

6421 ctatttgatg ttcaaataaa gaattaaact

What is claimed is:

1. A method for determining whether a compound modulates the protection an estrogen confers on a population of cells, the method comprising
a) exposing the cells to the compound
b) exposing the cells to the estrogen,
c) exposing the cells with an agent that damages the cells, and
d) evaluating the effect of estrogen on the cells in the presence of the compound, wherein a change in the effect of estrogen on the cells, relative to that within a population of cells that has not been exposed to the compound, indicates that the compound modulates the protection an estrogen confers on a population of cells.

2. The method of claim 1, wherein the estrogen is E₂, estrone, estriol, or a combination thereof.

3. The method of claim 1, wherein the molecule is a small molecule, peptide, or protein.

4. The method of claim 1, wherein the molecule is an agonist or antagonist of an estrogen receptor.

5. The method of claim 1, wherein the molecule is tamoxifen or an active metabolite thereof.

6. The method of claim 5, wherein the active metabolite is 4-hydroxytamoxifen (4-OH-Tam).

7. The method of claim 1, wherein the population of cells is a population of endothelial cells, ovarian cells, pulmonary cells, umbilical cells, or erythrocytes.

8. The method of claim 1, wherein the population of cells is a population of bovine aortic endothelial cells.

9. The method of claim 1, wherein the population of cells is a population of human aortic endothelial cells.

10. The method of claim 1, wherein the population of cells is a population of porcine endothelial cells, canine endothelial cells, equine endothelial cells, or human endothelial cells.

11. The method of claim 1, wherein the population of cells is a population of arterial or veinous cells.

12. The method of claim 1, wherein the population of cells is a population of cell types that expresses a recombinantly produced estrogen receptor.

13. The method of claim 1, wherein the estrogen is E₂, estrone, estriol, or a combination thereof.

14. The method of claim 1, wherein exposing the cells to the estrogen occurs before exposing the cells to either the molecule or the agent.

15. The method of claim 1, wherein exposing the cells to the estrogen and exposing the cells to the molecule occurs together.

16. The method of claim 1, wherein exposing the cells to the molecule occurs before exposing the cells to the estrogen.

17. The method of claim 1, wherein evaluating the cells comprises assaying cell survival.

18. The method of claim 1, wherein evaluating the cells comprises assaying the expression of an antioxidant enzyme.

19. The method of claim 17, wherein the antioxidant enzyme is a peroxidase.

20. The method of claim 17, wherein the antioxidant enzyme is glutathione peroxidase (GSHPX), catalase (CAT), superoxide dismutase (SOD), glutathione transferase (GSHTx), glutathione reductase (GR), glucose 6-phosphate dehydrogenase (G6PD), glutathione; ceruloplasmin, nitric oxide synthase, or GPX.

21. The method of claim 1, wherein the agent generates a reactive oxygen species in the cell.

22. The method of claim 1, wherein the effect is an attenuation of the effect of the estrogen.

23. The method of claim 1, wherein the attenuation is a reduction of at least 5% of the effect of the estrogen in absence of the molecule.

24. The method of claim 1, wherein the attenuation is a reduction of at least 50% of the effect of the estrogen in absence of the molecule.

25. The method of claim 1, wherein the attenuation is a reduction of at least 90% of the effect of the estrogen in absence of the molecule.

26. The method of claim 1, wherein the agent is paraquat, homocysteine, or lysophosphatidylcholine.

27. A method for determining whether a compound attenuates the protection an estrogen confers on a population of cells, the method comprising
a) exposing the cells to the compound and the estrogen,
b) treating the cells with an agent that generates a reactive oxygen species in the cells, and
c) evaluating survival of the cells, wherein a decrease in the number of surviving cells, relative to that within a population of cells that has not been exposed to the compound, indicates that the compound attenuates the protection an estrogen confers on a population of cells.

28. A method for determining whether a compound attenuates the protection an estrogen confers on a population of cells, the method comprising
a) exposing the cells to the compound and the estrogen,
b) treating the cells with an agent that generates a reactive oxygen species in the cells, and
c) evaluating survival of the cells, wherein an increase in the expression of an antioxidant enzyme, relative to the expression of the antioxidant enzyme within a population of cells that has not been exposed to the compound, indicates that the compound attenuates the protection an estrogen confers on a population of cells.

29. A method for determining whether a compound effects the protection an estrogen confers on a population of cells, the method comprising
a) exposing the cells to the compound and the estrogen,
b) exposing the cells with an agent that generates a reactive oxygen species in the cells, and
c) evaluating the expression of an antioxidant enzyme in the cells, wherein a change in the expression level of the antioxidant enzyme, relative to that within a population of cells that has not been exposed to estrogen and the compound, indicates that the compound effects the protection an estrogen confers on a population of cells.
